(19) United States
(12) Patent Application Publication
(10) Pub. No.: US 2013/0045898 A1
(43) Pub. Date: Feb. 21, 2013

(54) POLYESTER AMIDE FOAMERS

(71) Applicants: Stijn Witters, Lommel (BE); Franciscus Johannes Marie Derks, Heythuysen (NL)

(72) Inventors: Stijn Witters, Lommel (BE); Franciscus Johannes Marie Derks, Heythuysen (NL)

(73) Assignee: DSM IP ASSETS B.V., Heerlen (NL)

(21) Appl. No.: 13/512,229

(22) PCT Filed: Nov. 25, 2010
(86) PCT No.: PCT/EP2010/068201

§ 371 Date: Sep. 26, 2012

(30) Foreign Application Priority Data
Nov. 25, 2009 (EP) 09177039.6

(51) Int. Cl. C09K 8/94; C07C 9/00; E21B 43/34; C07C 233/63; C07D 413/14; E21B 43/00; C10G 99/00; C07C 233/47

(52) U.S. Cl. 507/202; 208/14; 585/16; 560/170; 560/169; 560/116; 544/87; 166/267

(57) ABSTRACT

There is disclosed use of hyperbranched polyester amides as a foamer to stabilize mixtures of oil and water especially for mixtures found in gas and/or oil wells to facilitate extraction of the well contents.

The present invention relates to the field of foamers such as those used to foam mixtures of water and oil and/or gas.

It is sometimes desirable to create foams of gas, water and oil. Examples of the use of such foams are for example in gas wells.

During gas mining, liquids are carried to the surface by the gas. These liquids cause a pressure drop by gravity and decrease gas velocity in the well bore in time. Four basic flow patterns can be distinguished, which are determined by the velocity of the gas and liquid phases but also the relative amount of gas and liquid at any given point in the flow stream. Liquid accumulates at the bottom of the well which slows down the gas production or can stop the gas production totally.

Deliquification of the (dead) well by surfactant injection (Foam Assisted Lift or FAL of water) is dependent on the critical velocity of the gas to carry liquid to the surface. A generally accepted model for the critical velocity to unload a gas well is based upon the mechanics of drop entrainment. The minimum velocity is defined by the rate at which the largest drop, that can exist, will move upward. The simple model is subject to a number of criticisms but captures the important physics of this process.

It is believed that the lifetime of a foam is controlled by a three stage process consisting of lamella (thin film formation), film thinning and rupture of the film where film thinning is the rate limiting step. The strength of a foam depends upon a complex relationship between the solution concentration of the surfactant and the surface activity of the foamer. In general, foaming requires a strongly heterogeneous interface. Therefore, oils are difficult to foam without the presence of an aqueous phase. A strongly heterogeneous interface means that the surface concentration of the surfactant is significantly in excess of the average concentration in solution.

For gas well deliquification, desirable foam is one that will produce the maximum amount of foam that can be handled in the process stream. Chemical defoamers are occasionally used in the separation equipment.

As early in 1959, Dunning et al tested different commercial foaming agents for removal of liquids from gas wells with the standard Ross-Miles method for the initial screening. Commercial surfactants that have been evaluated by this method as foamers for gas wells include:

Ethoxy surfactants (such as those available commercially under the trade designations Afrox or Armonist from Akzo Nobel); ethoxylated tall oils (such as those available commercially under the trade designation Ethofat from Akzo Nobel); alkyl mono ethanol amide ethoxylates (such as those available commercially under the trade designations Ethomid from Akzo Nobel); lauryl-beta-imino dipropionate (such as those available commercially under the trade designations Deriphat from General Mills or Joy from Proctor & Gamble); ethylene oxides (such as those available commercially under the trade designations Pluronic from Wyandotte Chemical); and ethylene oxide/propylene oxides (such as those available commercially under the trade designations Priminox or Triton from Rohm & Haas and Solar from Swift).

U.S. Pat. No. 4,237,977 (Skyline Products) describes a method for the production of a solid soap stick which can be used for gas well deliquification. The soap consists of urea, a water soluble non-ionic linear polyethoxylated surfactant and a lubricant to aid in the compression step. The surfactant type can be varied.

Another method for gas deliquification by solid soap stick addition is described in U.S. Pat. No. 5,515,924 (Glenn Osterhoudt) using a hydrogen producing, self reacting, self agitating solid foam stick. The soap stick, containing sodium borohydride, is dropped into a gas well together with a pH altering fluid. The fluid accelerates the reaction wherein a foam is created and removed, restoring a dead gas well.

Removal of particulates from a wellbore by foam formation is described by T. R. Thomas in U.S. Pat. No. 5,591,701. The foam is formed by a pH controlled process wherein an amphoteric foaming agent and an anionic or cationic surfactant are employed. Depending on hardness and salinity of the water, a selection can be made to form stable foams which can be collapsed. The patent teaches a preference for combining approximately equimolar ratios of amphoteric to anionic surfactants with small amounts of fatty acids or their salts to generate foam at high pH and then to defoam at low pH.

The chemicals tested in this foaming/defoaming process were combinations of acidic, basic and/or amphoteric surfactants such one or more of those listed below.

Acid surfactants such as: coco ampho carboxy propionic acid; acrylic acid/dimethyl di allyl ammonium chloride copolymer; coco trimethyl ammonium chloride; linear C₉₋₁₁ alcohol alkoxy sulfate; linear C₆₋₁₀ alcohol alkoxy sulfate; tri-ethylene glycol mono hexyl ether sulphate; C₁₄₋₁₆ alpha olefin sulphonate; ethylene diamine tetra-acetic acid tri-sodium salt; oleic acid; dodecanoic acid; coco acid; octodecanoic acid; lauric acid; myristic acid; hexadecanoic acid; and/or octanoic acid.

Basic surfactants such as: coco dimethyl amine oxide; coco amido propyl sulfo betaine; coco amido propyl betaine; coco amido propyl amino oxide; tallow bis-hydroxyethyl glycinate; coco diethanol amide; oleic acid diethanol amide; coco N,N bis-(hydroxyethyl)amide; ethoxylated coco amine; lauramine oxide; and/or dihydroxy ethyl C₁₂₋₁₅alkoxy propyl amine oxide.

Amphoteric surfactants such as: coco ampho acetate; coco ampho propionate, coco ampho hydroxypropyl sulphonate; lauro ampho acetate; lauro ampho dipropionate; capryl ampho diacetate; sodium lauriminopropionate; disodium tallow iminodipropionate; and/or stearo ampho propyl sulfonate.

Thus it is known that in more hydrophilic environments anionic surfactants (such as alkyl ether sulphates and olefin sulphonates) produce excellent foams. A disadvantage of this type of surfactant can be degradation.

When high gas condensate amounts are present, cationic surfactants like quaternary amines perform very well. However, high molecular weight surfactants can be ineffective in brines and they are all demonstrated to cause potential emulsion issues if over treated. Amphoteric surfactants have been considered the most versatile type of foamers. They are stable at high temperatures (>200° F.) and tolerant of high salt content (+10 w %).

U.S. Pat. No. 7,422,064 describes branched and linear C₃₋₁₈alcohols in foam forming compositions. No star shaped/hyperbranched/dendritic surfactants are described.

US 2006-0229231-A describes betaines and quaternary amines with linear and branched structures.

Though surfactants for gas well deliquification have been used for a long time, historically they have been solid foamers applied in the form of soap sticks or solid state products (urea in combination with ethoxylated surfactants) that are dropped down the well bore. For solid foamers there has been an issue ensuring that the chemical reached the well region where the liquid was located.

To overcome this problem new liquid foamers have been introduced customized to function in particular gas well environments. Surfactants of different types have been used such as non-ionic (polyethoxylated compounds of phenols or alcohols), anionic (alkyl ether sulphates and alpha-olefin sulphonates), cationic (quaternary amines) and amphoteric (dual charge).

None of these surfactants is entirely satisfactory as a foamer especially for use in gas wells more especially where there is a high brine content in the well.

It is a preferred object of the invention to solve some or all of the problems identified herein.

Surprisingly the applicant has discovered that polyester amides, preferably hyperbranched polyester amides are useful in forming stable foams from mixtures of gas, water and oil. They are particularly useful in rapidly forming stable foams from mixtures of brine and oil which have previously resisted foaming with other common foaming agents.

Therefore broadly in accordance with the present invention there is provided a foam formed from a mixture of an organic phase (such as gas and/or oil) and an aqueous phase (such as brine) where the foam is stabilised by a polyester amide, preferably a hyperbranched polyester amide.

As used herein unless the context dictates otherwise the term “oil” means oil and condensate such as for example crude oil found in for example subterranean deposits and extracted by for example drilling bore holes; and the term “gas” means for example the natural gas found and extracted in a similar manner to oil (often at the same time). Similar materials extracted by similar or analogous methods are also encompassed in the scope of this invention.

In one embodiment of the invention the foam may comprise at least one other surfactant and/or synergist (i.e. in addition to the polyester amide, such as any of those known surfactants described herein).

Another aspect of the invention broadly provides a use of a foaming agent comprising polyester amide (preferably hyperbranched polyester amide) to stabilize a foam (such as those of the invention).

A further aspect of the invention broadly provides a method for forming, from a mixture of an organic phase an aqueous phase (such as an [oil and/or gas] and [water or brine] mixture) a foam (such as those of the invention), where the method comprising the step of adding a polyester amide (preferably hyperbranched polyester amide) to the mixture to form the foam.

A still other aspect of the invention broadly provides for a method of manufacture of a polyester amide (preferably hyperbranched polyester amide) for the purpose of forming, from a mixture of an aqueous phase and an organic phase (such as an [oil and/or gas] and [water or brine] mixture) a foam (such as those of the invention).

A yet still other aspect of the invention broadly provides for a method for extracting an organic substance from a bore hole and/or well comprising the steps of:
adding polyester amide (preferably hyperbranched polyester amide) to the bore to form, (such as an [oil and/or gas] and [water or brine] mixture) a foam (such as those of the invention);
transporting the foam from the bore hole and/or well;
destabilizing the foam to separate into an organic and an aqueous phase; and optionally collecting the organic substance.

Preferred methods of extraction are from a gas or oil well and where the organic substance is gas or oil.

A further yet still other aspect of the invention broadly provides for use of a polyester amide (preferably hyperbranched polyester amide) in any of the methods of the invention described herein.

A more further yet still other aspect of the invention broadly provides an organic substance (such as gas and oil) obtained and/or obtainable by a method of extracting organic substance (such as oil/gas) as described above.

Creation of the foam and the stability thereof may be tested as described herein in Foam test 1. Preferred polyester amides for use herein may be those that produce foams stable under the harsh conditions reproduced in Foam test 2 which are analogous to those faced in the underground reservoir of a typical gas well.

Preferably the polyester amides foamers used herein are substantially non-linear, non-cyclic branched macromolecules (such as polymers) having three or more polymeric centres, more preferably having a molecular weight of at least 100. Usefully the polyester amides are three dimensional hyperbranched polymers, star-shaped polymers or dendrimeric macro-molecules.

Conveniently the polyester amide foamers useful in the present invention comprise one or more polar moieties and one or more apolar moieties optionally disposed around the periphery of the macromolecule(s).

Suitable polar groups are selected from: hydroxy, carboxy, optionally alkyl substituted imino, polyalkoxy chains; any other groups described herein as moieties; and/or suitable combinations and/or mixtures thereof.

Suitable apolar groups may be optionally substituted hydrocarbo groups comprising at least 4 carbon atoms.

Preferred polyester amide foamers useful in the present invention comprise those in which the (average) ratio of polar groups to apolar groups is from about 0.01 to about 20, more preferably from 0.05 to 10, most preferably from 0.1 to 8.0, for example from 0.13 to 7.75 such as 0.13, 0.50, 0.57, 0.64, 1.00, 1.90, 7.00 and/or 7.75. These ratios may be weight ratios and/or molar ratios, preferably are weight ratios.

Where the polyester amides foamers are hyper-branched polymers they may be prepared by the methods described one or more of the publications below (and combinations thereof) and/or have structures as described thereto. The contents of these documents are incorporated by reference.

Hydroxyl functional hyper-branched polyester amide polymers are described in WO 1999-016810.

Carboxy functional hyperbranched polyester amide polymers are described in WO 2000-056804.

Dialkyl amide functional hyperbranched polyester amide polymers are described in WO 2000-058388.

Ethoxy functional hyperbranched polyester amide polymers are described in WO 2003-037959.

Hetero functionalised hyperbranched polyester amides are described in WO 2007-090009.

Secondary amide hyperbranched polyester amides are described in WO 2007-144189.

Hyperbranched polyester amides that may be used in the present invention may be water soluble and optionally soluble in most organic solvents.

Preferred hyperbranched polyester amides of use in the present invention may obtained and/or obtainable from: at least one organo building block and at least one tri (or higher) organo valent branching unit, where the at least one building block is capable of reacting with the at least one branching unit; and at least one or the building block and/or the branching unit (conveniently the branching unit) comprises an end group comprising a polar moiety.

More preferred hyperbranched polyester amides of use in the present invention may obtained and/or obtainable from: at least one building block comprising one or more polycarboxylic acid(s) and/or one or more anhydride(s) obtained and/or obtainable from one or more polycarboxylic acid(s); and

at least one branching unit comprising at least one tri functional nitrogen atom where the at least one branching unit containing an end group comprising a polar moiety.

Suitable polycarboxylic acid(s) that be used as and/or to prepare the building block(s) may conveniently be dicarboxylic acids such as C₂₋₁₂hydrocarbo dicarboxylic acids; more conveniently linear di-acids and/or cyclic di-acids; and most conveniently linear di-acids with terminal carboxylic acid groups such as those selected from the group consisting of: saturated di-acids such as:

2-ethanedioic acid (oxalic acid); 3-propanedioic acid (malonic acid); 4-butanedioic acid (succinic acid), 5-pentanedioic acid (glutaric acid); 6-hexanedioic acid (adipic acid); 7-heptanedioic acid (pimelic acid); 8-octanedioic acid (suberic acid); combinations thereof; and mixtures thereof; and
unsaturated di-acids such as:
Z-(cis)-butenedioic acid (maleic acid); E-(trans)-butenedioic acid (fumaric acid); 2,3-dihydroxybutandioic acid (tartaric acid); combinations thereof; and mixtures thereof.

Suitable branching units may be any moiety capable of reacting with the building block at three or more sites on the branching unit to form a three dimensional (branched) product.

Branching units may comprise one or more polyoxyalkylene moiet(ies) comprises polyoxyalkylene repeat unit(s) for example suitable unsubstituted or substituted alkylene groups such as ethylene, propylene, butylene, and isobutylene. The polyoxyalkylene moiety comprising one or more of these repeat units can be a homo-, block or random polymer, or any suitable mixtures thereof. Preferred the average total number of repeat units in polyoxyalkylene moiet(ies) suitable for use in branching units herein is from 2 to 100, more preferably 5 to 60, most preferably 10 to 50, for example 16 or 45.

Useful hyperbranched polyester amides of use in the present invention may obtained and/or obtainable from:

at least one building block selected from the group consisting of:

optionally substituted C₂₋₃₀hydrocarbo dioic acid,

anhydrides thereof;

combinations thereof on the same moiety; and

mixtures thereof on different moieties;

at least one branching unit selected from the group consisting of:

optionally substituted C₁₋₂₀hydrocarbo amine;

combinations thereof on the same moiety; and

mixtures thereof on different moieties;

where the at least one branching unit comprises at least one (additional) polar end group optionally selected from one or more:
optionally neutralised carboxylic acid or groups derived therefrom;

optionally terminated poly(alkyenyloxy) groups

optionally quaternized hydrocarbo substituted amino

optionally substituted hetero atom containing rings;

secondary hydroxyl groups;

More useful hyperbranched polyester amides of use in the present invention may obtained and/or obtainable from: at least one building block selected from the group consisting of:

C₄₋₁₆alkenyl C₂₋₁₀dioic anhydride;

C₄₋₁₆cycloalkyl dicarboxylic acid anhydride;

C₂₋₁₀alkandioic anhydride;

combinations thereof on the same moiety; and

mixtures thereof on different moieties;

at least one branching unit selected from the group consisting of:

optionally hydroxyl substituted di-C₁₋₈hydrocarbyl secondary amine;

combinations thereof on the same moiety; and

mixtures thereof on different moieties;

where the at least one branching unit comprises at least one end group selected from the group consisting of:
optionally neutralised carboxylic acid groups;
poly(C₂₋₄alkyenyloxy), groups optionally terminated with an alkoxy group (e.g. C₁₋₄alkoxy) where optionally n is from 15 to 50;
optionally quaternized C₁₋₁₂hydrocarbo (e.g. C₁₋₆hydrocarbyl) substituted amino optionally substituted nitrogen containing C₃₋₁₀ rings (such as morpholo);
secondary hydroxyl groups;
combinations thereof on the same moiety; and
mixtures thereof on different moieties.

Most useful hyperbranched polyester amides of use in the present invention may obtained and/or obtainable from:

at least one building block selected from the group consisting of:

dodecenyl (i.e. Cᵤalkenyl) succinic (i.e. 4-butanedioic) anhydride;

cyclohexane-1,2-dicarboxylic acid anhydride;

succinic (i.e. 4-butanedioic) anhydride;

combinations thereof on the same moiety; and

mixtures thereof on different moieties;

at least one branching unit selected from the group consisting of:

diisopropanol amine; diethanolamine;

trishydroxymethylene amino methane;

combinations thereof on the same moiety; and

mixtures thereof on different moieties;

where the at least one branching unit comprises at least one end group selected from the group consisting of:
carboxylic acid groups optionally neutralized with ammonia;
methoxy-terminated polyethylene glycol (molecular weight from 350 to 10000) [tertiary amine groups; optionally quaternized with dimethyl sulphate or protonized with acids morpholine;
secondary hydroxyl groups;
combinations thereof on the same moiety; and
mixtures thereof on different moieties.

Advantageously hyperbranched polyester amides of use in the present invention may have a (theoretical) number average molecular weight (Mₙ) of from about 500 to about 50000 g/mol; more advantageously from about 800 to about 30000 g/mol; most advantageously from about 1000 to about 20000 g/mol; even more particularly from about 1200 to about 17000 g/mol; for example selected from at least one molecular weight from the group consisting of: 1200 g/mol, 1600 g/mol; 1900 g/mol; 4000 g/mol; 4200 g/mol; 4400 g/mol; 15000 g/mol; and 17000 g/mol;

Optionally convenient hyperbranched polyester amides of use in the present invention have one or more of the following properties: at least 50% solids, for example 50% solids in 45% H₂O and 5% ethanol;

Specific examples of structures of particular preferred hyperbranched polyester amide suitable for use as foamers, especially for use as foamers for gas/oil wells are given below.

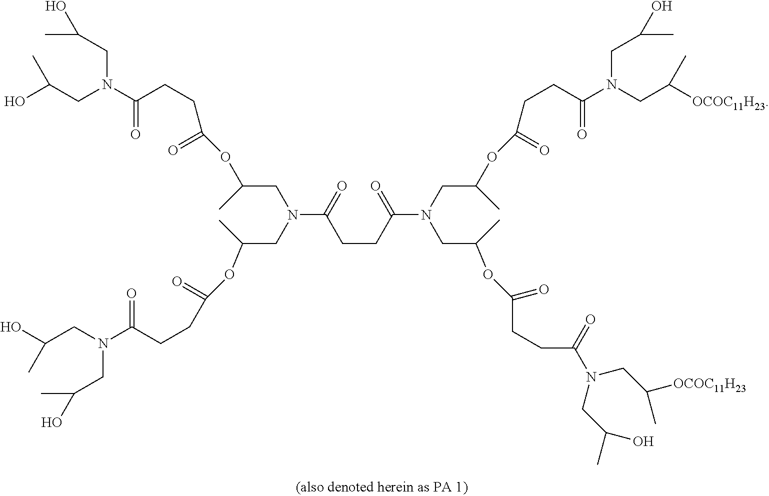

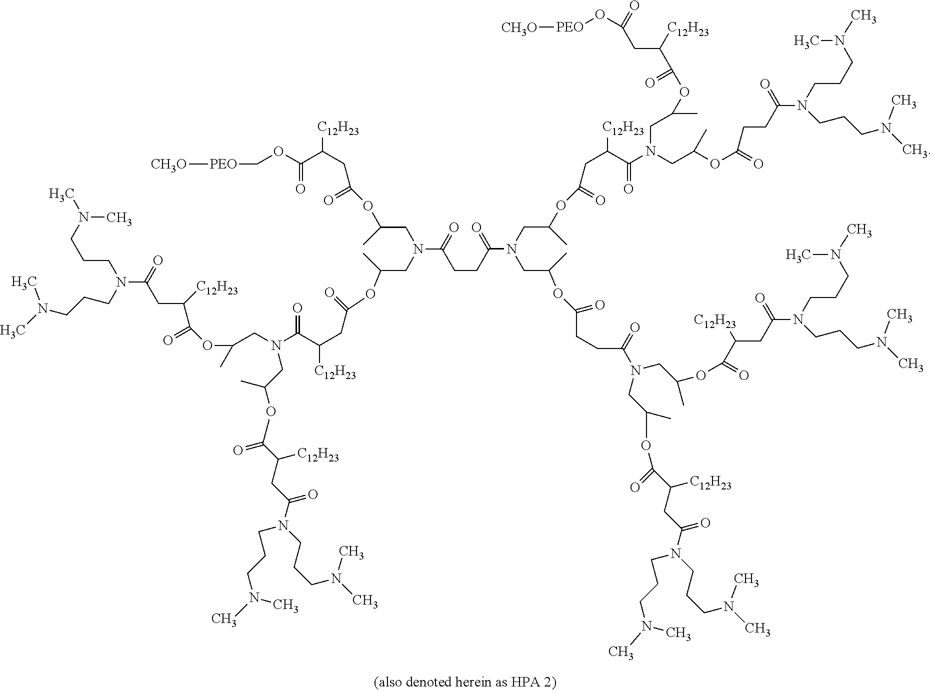

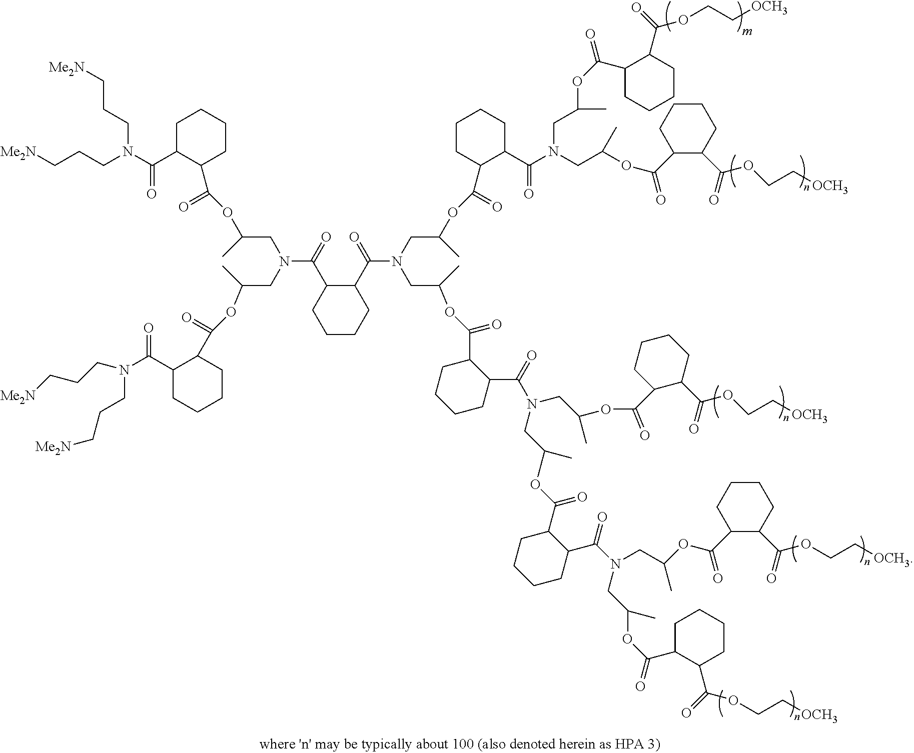

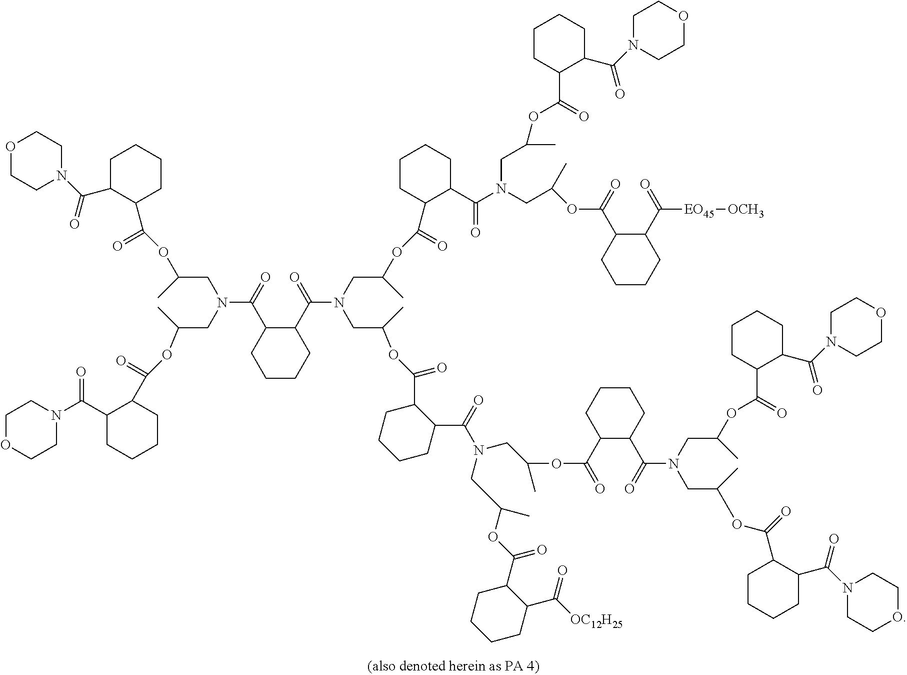

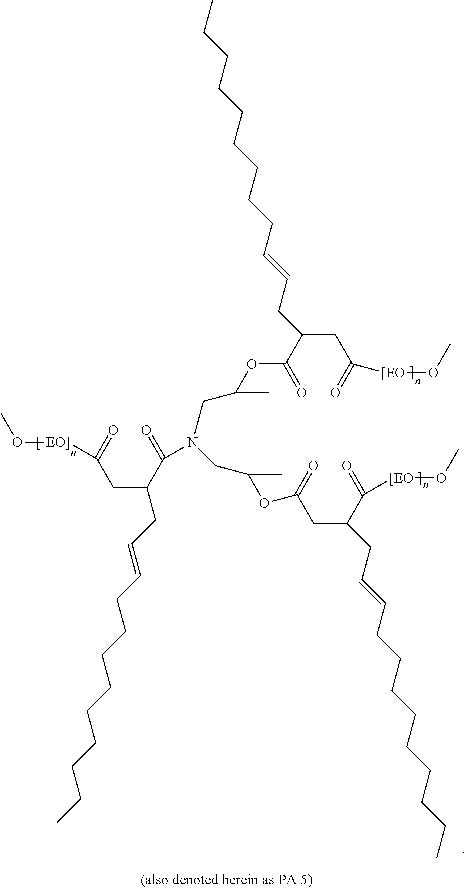

Those water soluble hyperbranched polyester amides obtained and/or obtainable from a dodecenyl succinic anhydride building block with a diisopropanol amine branching unit, having carboxylic acid end groups neutralized with ammonia and a (theoretical): number average molecular weight of 4000 g/mol. A preferred typical idealized structure of such hyperbranched polyester amides is:

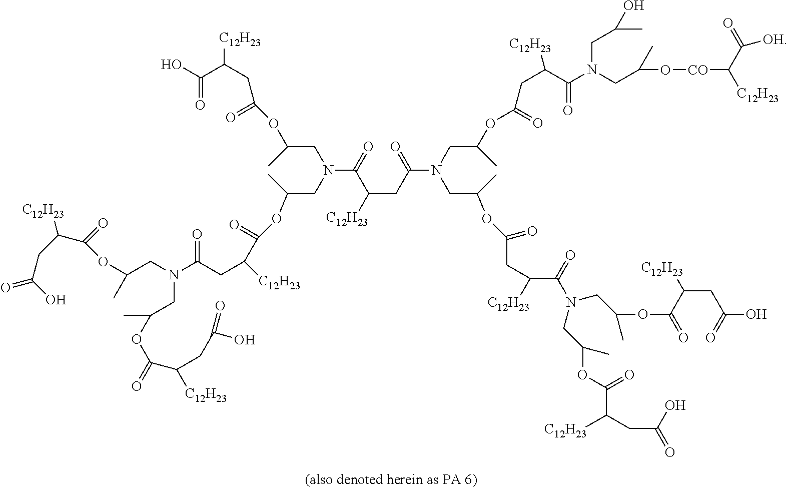

Those water soluble (and soluble in most organic solvents) hyperbranched polyester amides obtained and/or obtainable from a dodecenyl succinic anhydride building block with a diisopropanol amine branching unit, having end groups of methoxy-terminated polyethylene glycol (molecular weight=2000) and a (theoretical): number average molecular weight of 17000 g/mol. A preferred typical idealized structure of such hyperbranched polyester amides is:

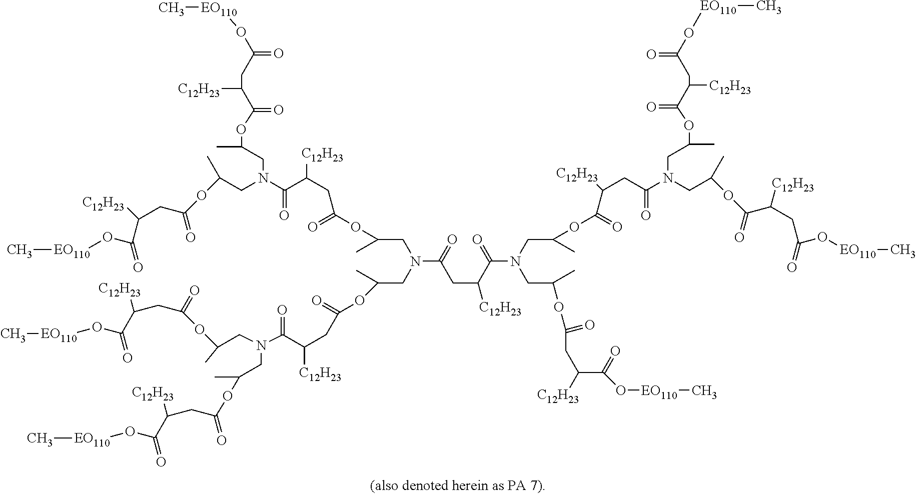

Those water soluble (and soluble in most organic solvents) hyperbranched polyester amides obtained and/or obtainable from a dodecenyl succinic anhydride and succinic anhydride building blocks with a diisopropanol amine branching unit, having end groups of (about 90%) of 3,3′-iminobis(N,N-dimethylpropyl amine) quaternized with dimethyl sulfate and (about 10%) of methoxy-terminated polyethylene glycol (molecular weight 750); and a (theoretical): number average molecular weight of 4200 g/mol. A preferred typical idealized structure of such hyperbranched polyester amides is:

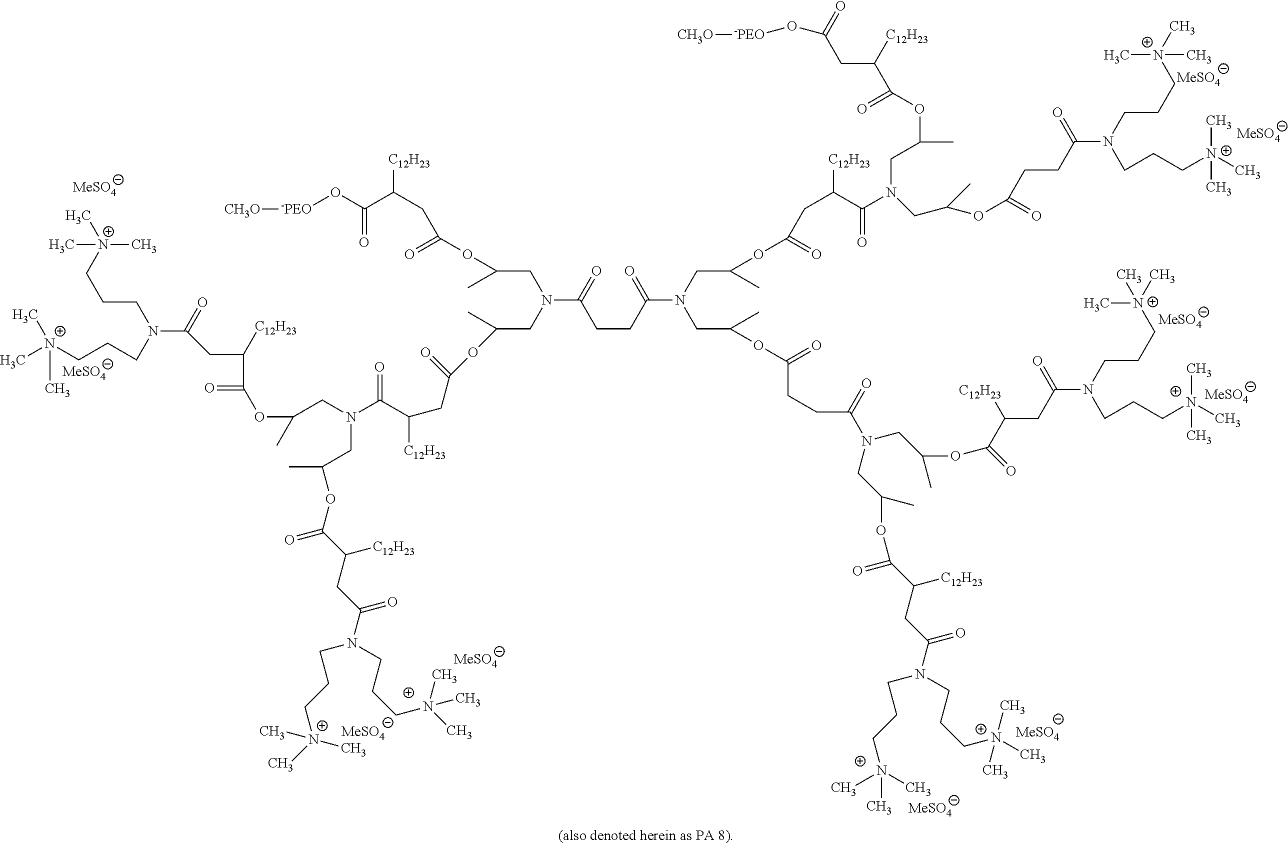

Those water soluble hyperbranched polyester amides obtained and/or obtainable from a cyclohexane-1,2-dicarboxylic acid anhydride building block with a diisopropanol amine branching unit, having end groups of (about 90%) of tertiary amine; and a (theoretical): number average molecular weight of 1600 g/mol. A preferred typical idealized structure of such hyperbranched polyester amides is:

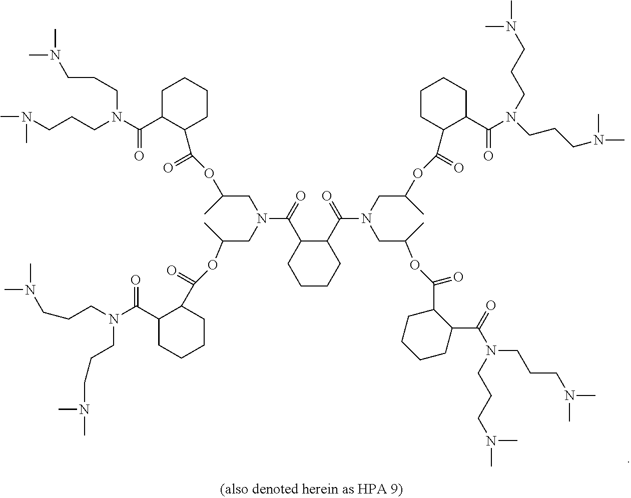

Those water soluble hyperbranched polyester amides obtained and/or obtainable from a cyclohexane-1,2-dicarboxylic acid anhydride building block with a diisopropanol amine branching unit, having end groups of (about 90%) of quaternary amine quaternised with dimethyl sulfate; and a (theoretical): number average molecular weight of 1600 g/mol. A preferred typical idealized structure of such hyperbranched polyester amides is:

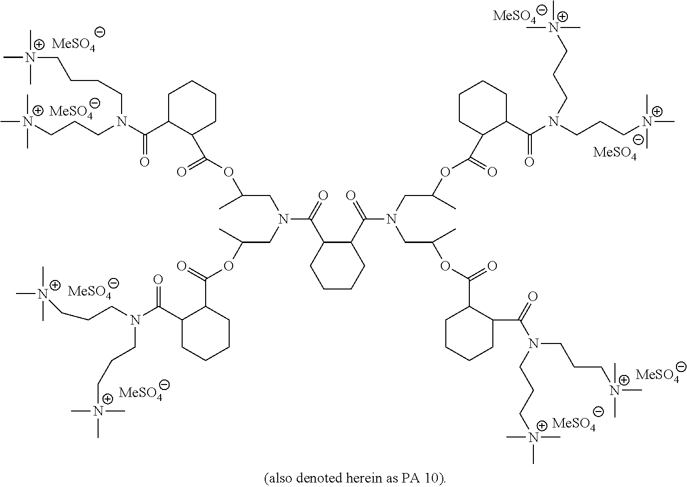

Those water soluble (and soluble in most organic solvents) hyperbranched polyester amides obtained and/or obtainable from a cyclohexane-1,2-dicarboxylic acid anhydride building block with a diisopropanol amine branching unit, having end groups of (about 60%) morpholine and (optionally about 40%) of methoxy-terminated polyethylene glycol with about 16 ethoxy (EO) units (molecular weight of 750); and a (theoretical): number average molecular weight of 4400 g/mol. A preferred typical idealized structure of such hyperbranched polyester amides is:

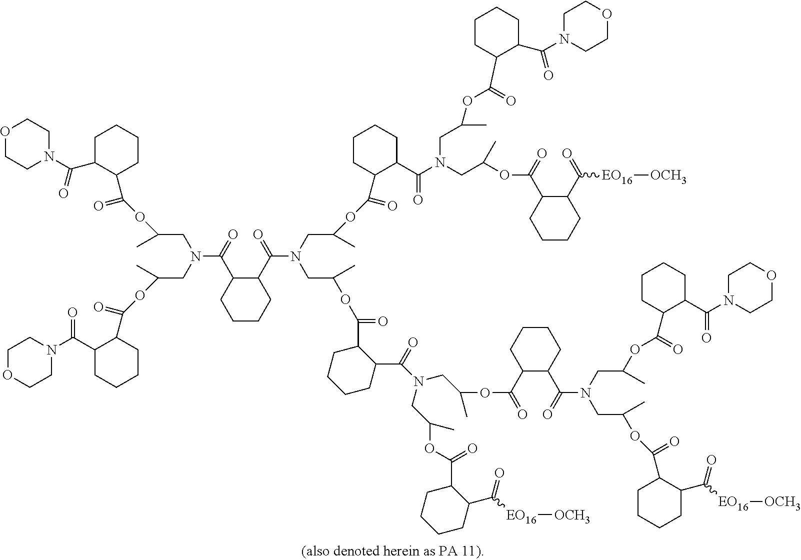

Those water soluble (and soluble in most polar organic solvents) hyperbranched polyester amides obtained and/or obtainable from a succinic anhydride building block with a diisopropanol amine branching unit, having secondary hydroxyl end groups; and a (theoretical) number average molecular weight of 1200 g/mol. A preferred typical idealized structure of such hyperbranched polyester amides is:

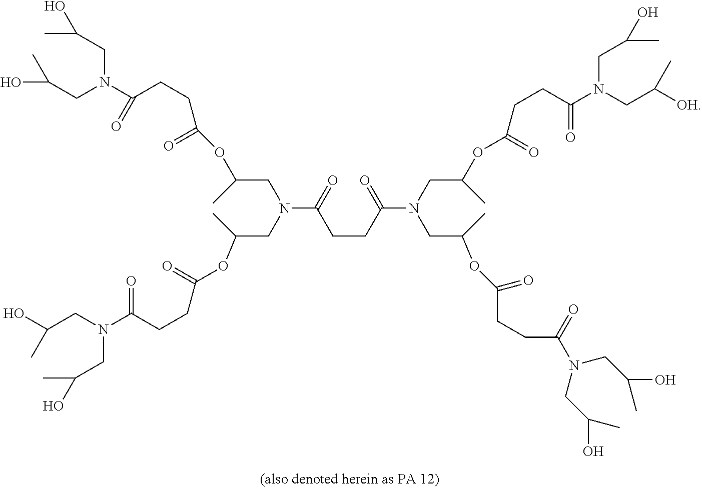 [SMILES: CC(O)CN(CC(C)O)C(=O)CCC(=O)OC(C)CN(CC(C)OC(=O)CCC(=O)N(CC(C)O)CC(C)O)C(=O)CCC(=O)N(CC(C)OC(=O)CCC(=O)N(CC(C)O)CC(C)O)CC(C)OC(=O)CCC(=O)N(CC(C)O)CC(C)O]

Those water soluble hyperbranched polyester amides obtained and/or obtainable from a succinic anhydride building block with a trishydroxymethylene amino methane branching unit, having [optionally about 95% of] tertiary amine end groups; and a (theoretical) number average molecular weight of 1900 g/mol. A preferred typical idealized structure of such hyperbranched polyester amides is:

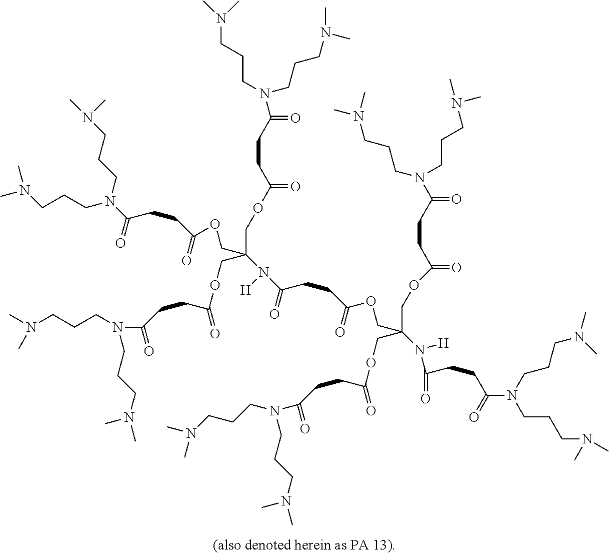

Those water soluble (and soluble in most organic solvents) hyperbranched polyester amides obtained and/or obtainable from a succinic anhydride building block with a diisopropanol amine branching unit, having end groups of methoxy-terminated polyethylene glycol with about 45 EO units (molecular weight of 2000); and a (theoretical) number average molecular weight of 15000 g/mol. A preferred typical idealized structure of such hyperbranched polyester amides is:

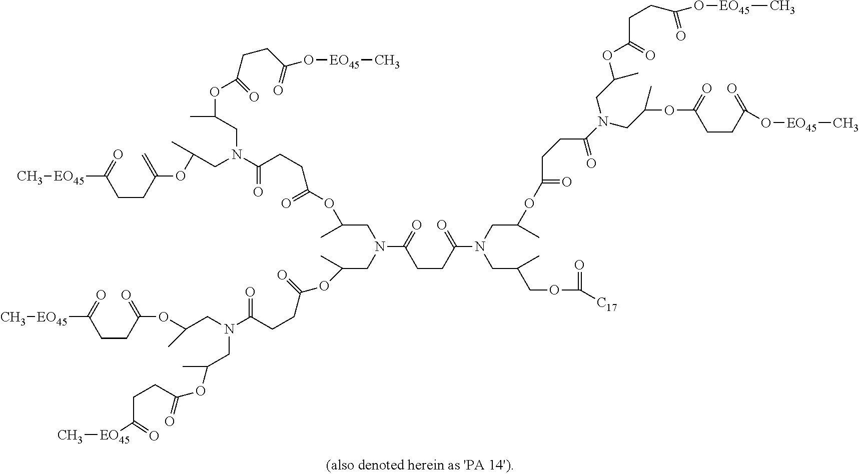

The terms ‘optional substituent’ and/or ‘optionally substituted’ as used herein (unless followed by a list of other substituents) signifies the one or more of following groups (or substitution by these groups): carboxy, sulfo, sulfonyl, phosphates, phosphonates, phosphines, formyl, hydroxy, amino, imino, nitrilo, mercapto, cyano, nitro, methyl, methoxy and/or combinations thereof. These optional groups include all chemically possible combinations in the same moiety of a plurality (preferably two) of the aforementioned groups (e.g. amino and sulfonyl if directly attached to each other represent a sulfamoyl group). Preferred optional substituents comprise: carboxy, sulfo, hydroxy, amino, mercapto, cyano, methyl, halo, trihalomethyl and/or methoxy, more preferred being methyl, hydroxyl, amino and/or cyano, most preferred being hydroxyl and/or amino.

The synonymous terms ‘organic substituent’ and “organic group” as used herein (also abbreviated herein to “organo”) denote any univalent or multivalent moiety (optionally attached to one or more other moieties) which comprises one or more carbon atoms and optionally one or more other heteroatoms. Organic groups may comprise organoheteryl groups (also known as organoelement groups) which comprise univalent groups containing carbon, which are thus organic, but which have their free valence at an atom other than carbon (for example organothio groups). Organic groups may alternatively or additionally comprise organyl groups which comprise any organic substituent group, regardless of functional type, having one free valence at a carbon atom. Organic groups may also comprise heterocyclyl groups which comprise univalent groups formed by removing a hydrogen atom from any ring atom of a heterocyclic compound: (a cyclic compound having as ring members atoms of at least two different elements, in this case one being carbon). Preferably the non carbon atoms in an organic group may be selected from: hydrogen, halo, phosphorus, nitrogen, oxygen, silicon and/or sulphur, more preferably from hydrogen, nitrogen, oxygen, phosphorus and/or sulphur.

Most preferred organic groups comprise one or more of the following carbon containing moieties: alkyl, alkoxy, alkanoyl, carboxy, carbonyl, formyl and/or combinations thereof; optionally in combination with one or more of the following heteroatom containing moieties: oxy, thio, sulphinyl, sulphonyl, amino, imino, nitrilo and/or combinations thereof. Organic groups include all chemically possible combinations in the same moiety of a plurality (preferably two) of the aforementioned carbon containing and/or heteroatom moieties (e.g. alkoxy and carbonyl if directly attached to each other represent an alkoxycarbonyl group).

The term ‘hydrocarbo group’ as used herein is a sub set of a organic group and denotes any univalent or multivalent moiety (optionally attached to one or more other moieties) which consists of one or more hydrogen atoms and one or more carbon atoms and may comprise one or more saturated, unsaturated and/or aromatic moieties. Hydrocarbo groups may comprise one or more of the following groups. Hydrocarbyl groups comprise univalent groups formed by removing a hydrogen atom from a hydrocarbon (for example alkyl). Hydrocarbylene groups comprise divalent groups formed by removing two hydrogen atoms from a hydrocarbon, the free valencies of which are not engaged in a double bond (for example alkylene). Hydrocarbylidene groups comprise divalent groups (which may be represented by “R₂C═”) formed by removing two hydrogen atoms from the same carbon atom of a hydrocarbon, the free valencies of which are engaged in a double bond (for example alkylidene). Hydrocarbylidyne groups comprise trivalent groups (which may be represented by “RC≡”), formed by removing three hydrogen atoms from the same carbon atom of a hydrocarbon the free valencies of which are engaged in a triple bond (for example alkylidyne). Hydrocarbo groups may also comprise saturated carbon to carbon single bonds (e.g. in alkyl groups); unsaturated double and/or triple carbon to carbon bonds (e.g. in respectively alkenyl and alkynyl groups); aromatic groups (e.g. in aryl groups) and/or combinations thereof within the same moiety and where indicated may be substituted with other functional groups

The term ‘alkyl’ or its equivalent (e.g. ‘alk’) as used herein may be readily replaced, where appropriate and unless the context clearly indicates otherwise, by terms encompassing any other hydrocarbo group such as those described herein (e.g. comprising double bonds, triple bonds, aromatic moieties (such as respectively alkenyl, alkynyl and/or aryl) and/or combinations thereof (e.g. aralkyl) as well as any multivalent hydrocarbo species linking two or more moieties (such as bivalent hydrocarbylene radicals e.g. alkylene).

Any radical group or moiety mentioned herein (e.g. as a substituent) may be a multivalent or a monovalent radical unless otherwise stated or the context clearly indicates otherwise (e.g. a bivalent hydrocarbylene moiety linking two other moieties). However where indicated herein such monovalent or multivalent groups may still also comprise optional substituents. A group which comprises a chain of three or more atoms signifies a group in which the chain wholly or in part may be linear, branched and/or form a ring (including spiro and/or fused rings). The total number of certain atoms is specified for certain substituents for example C_{1-N}organo, signifies a organo moiety comprising from 1 to N carbon atoms. In any of the formulae herein if one or more substituents are not indicated as attached to any particular atom in a moiety (e.g. on a particular position along a chain and/or ring) the substituent may replace any H and/or may be located at any available position on the moiety which is chemically suitable and/or effective.

Preferably any of the organo groups listed herein comprise from 1 to 36 carbon atoms, more preferably from 1 to 18. It is particularly preferred that the number of carbon atoms in an organo group is from to 12, especially from 1 to 10 inclusive, for example from 1 to 4 carbon atoms.

As used herein chemical terms (other than IUAPC names for specifically identified compounds) which comprise features which are given in parentheses—such as (alkyl)acrylate, (meth)acrylate and/or (co)polymer denote that that part in parentheses is optional as the context dictates, so for example the term (meth)acrylate denotes both methacrylate and acrylate.

Certain moieties, species, groups, repeat units, compounds, oligomers, polymers, materials, mixtures, compositions and/or formulations which comprise and/or are used in some or all of the invention as described herein may exist as one or more different forms such as any of those in the following non exhaustive list: stereoisomers (such as enantiomers (e.g. E and/or Z forms), diastereoisomers and/or geometric isomers); tautomers (e.g. keto and/or enol forms), conformers, salts, zwitterions, complexes (such as chelates, clathrates, crown compounds, cyptands/cryptades, inclusion compounds, intercalation compounds, interstitial compounds, ligand complexes, organometallic complexes, non stoichiometric complexes, π adducts, solvates and/or hydrates); isotopically substituted forms, polymeric configurations [such as homo or copolymers, random, graft and/or block polymers, linear and/or branched polymers (e.g. star and/or side branched), cross linked and/or networked polymers, polymers obtainable from di and/or tri valent repeat units, dendrimers, polymers of different tacticity (e.g. isotactic, syndiotactic or atactic polymers)]; polymorphs (such as interstitial forms, crystalline forms and/or amorphous forms), different phases, solid solutions; and/or combinations thereof and/or mixtures thereof where possible. The present invention comprises and/or uses all such forms which are effective as defined herein.

Polyester amides may also usefully exhibit other properties to be useful as an improved foamer. For example the polyester amides may exhibit at least one of those desired properties described herein (such as those below) and/or any combinations thereof that are not mutually exclusive.

Useful polyester amide(s) may exhibit one or more improved propert(ies) (such as those described herein) with respect to known foamers. More usefully such improved properties may be in a plurality, most usefully three or more of those properties below that are not mutually exclusive.

Conveniently the polyester amide(s) may exhibit one or more comparable propert(ies) (such as those described herein) with respect to known foamers. More usefully such comparable properties may be in two or more, most usefully three or more, for example all of those properties below that are not improved and/or mutually exclusive.

Improved propert(ies) as used herein denotes that the value of one or more parameter(s) of the foamer of the present invention is >+8% of the value of that parameter for the reference foam described herein, more preferably >+10%, even more preferably >+12%, most preferably >+15%.

Comparable properties as used herein means the value of one or more parameter(s) of the foam of the present invention is within +/−6% of the value of that parameter for the reference foamer described herein, more preferably +/−5%, most preferably +/−4%.

The known reference foamer for these comparisons are sodium lauryl sulphate (SLS) used in the same amounts as the foamer of the invention being compared.

The percentage differences for improved and comparable properties herein refer to fractional differences between the foamer of the invention and the known the foamer where the property is measured in the same units in the same way (i.e. if the value to be compared is also measured as a percentage it does not denote an absolute difference).

It is preferred that polyester amides of the invention (more preferably hyperbranched polyester amides) have an improved CMC value and/or surface tension (compared to SLS).

Without wishing to be bound by any mechanism, the applicant believes that thinning of the foam film is related to the critical micelle concentration (CMC). The CMC is the point at which the addition of surfactant to a solution results in the formation of colloidal aggregates. The more micelles present in solution, the easier the film ordering. The foamer with the lower CMC would, at the same concentration, have more micelles present and be more stable.

The following factors may contribute to a CMC decrease: an increase in the number of carbon atoms in the hydrophobic tails; the existence of polyoxypropylene group; fluorocarbon structure; an increased degree of binding of the counterions; the addition of electrolyte to ionic surfactants; the existence of polar organic compounds (such as alcohols and amides) and/or the addition of xylose and fructose

The following factors may contribute to a CMC increase: a more branched hydrophobic structure; double bonds between carbon atoms; polar groups (O or OH) in hydrophobic tail; strongly ionized polar groups (sulphates and quaternaries); hydrophilic groups placed in the surfactant molecule centre; increase in the number of hydrophilic head; trifluoromethyl groups; an increase in the effective size of the hydrophilic head; an increase in the pH of weak acids (such as soap); a decrease in pH from isoelectric region and increase in pH from isoelectric region for amphoteric surfactants (low CMC at the isoelectric region and high CMC outside the isoelectric region); and/or addition of urea, formamide, guanidinium salts, dioxane, ethylene glycol and water soluble esters

The film stability also is affected by electrolytes. The more salt present, the less ordering in the film which has a negative effect on the film stability. In addition, salt changes the CMC of the foamers, altering their efficacy. Impurities like solubilised oil or small solid particles will destabilize the foam by destruction of the intrinsic ordering of the lamella.

It will be appreciated that optionally if necessary the hyperbranched polyesters described herein may usefully be used in the methods of the invention in combination with other compatible surface active agents (such as any described herein) to form improved foams (as evaluated by one or more of the tests and/or criteria described herein).

Many other variations embodiments of the invention will be apparent to those skilled in the art and such variations are contemplated within the broad scope of the present invention.

Further aspects of the invention and preferred features thereof are given in the claims herein.

### Surface Tension

Surface tension is a convenient parameter to evaluate the theoretical basis for foam formation and for the prediction of minimum gas velocity. It is a function of the diffusion rate of the surfactant and is dependent upon the method of measurement. To gauge the dynamic surface tension of various foaming agents, experiments are performed based on the maximum bubble pressure method.

It is believed that surface activity (as indicated by a decrease in surface tension) appears to be useful for effective foam formation, although it certainly is not the only requirement.

Hyperbranched polyester amides can act as surfactants. Their highly branched or hyperbranched structure and high functionality gives them multiple polar and multiple apolar groups.

A high activity at lower concentrations results in a lower critical micelle concentration (CMC). In this way, it is believed that hyperbranched polyester amides can act as an improved surfactants, particularly suitable for use as a foamer for gas wells, as shown schematically in FIG. 3. Also shown in the right side of FIG. 3 is a possible mechanism of how hyperbranched polyester amides might act as a synergist between different interfaces (gas-liquid-gas) rather than forming lamellar layers of typical surfactants. Without being bound by any mechanism the applicant believes this may explain why hyperbranched polyester amides may be used (optionally in combination with conventional surfactants) as a surfactant and/or synergist to enhance foaming properties.

### FIGURES

The invention is illustrated by the following non-limiting figures where:

FIG. 1 shows typical flow patterns in vertical multi-phase flow gas well.

FIG. 2 shows progression of a gas water mixture a typical gas well over time.

FIG. 3 shows schematically how the dispersive mechanism of hyperbranched polyester amide surfactants (denoted by the trade mark ‘Hybrane’ in this figure) may differ from common surfactants.

FIG. 4 shows the effect of different concentrations of different dissolved hyperbranched polyester amides on the surface tension (used as a representative parameter for surface activity).

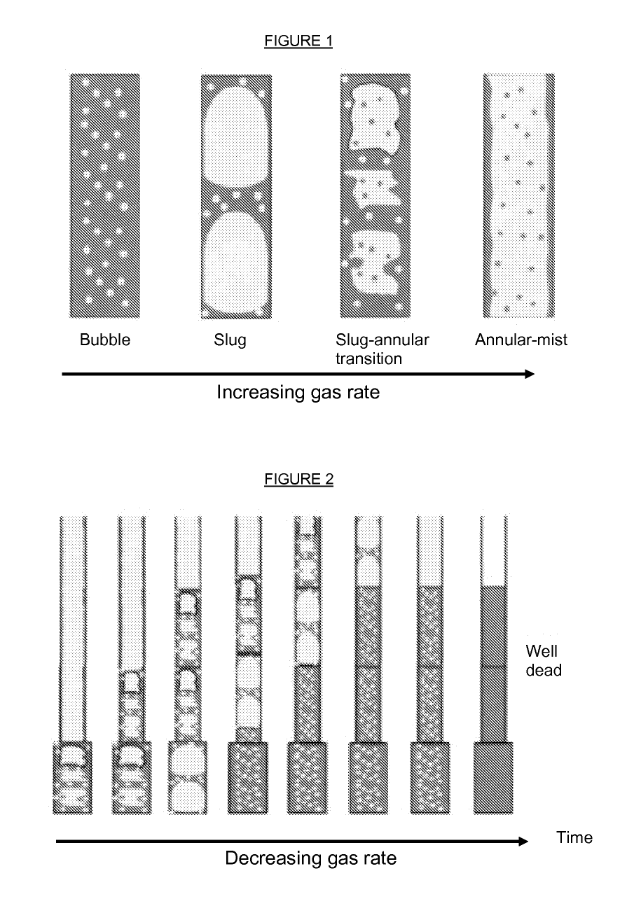

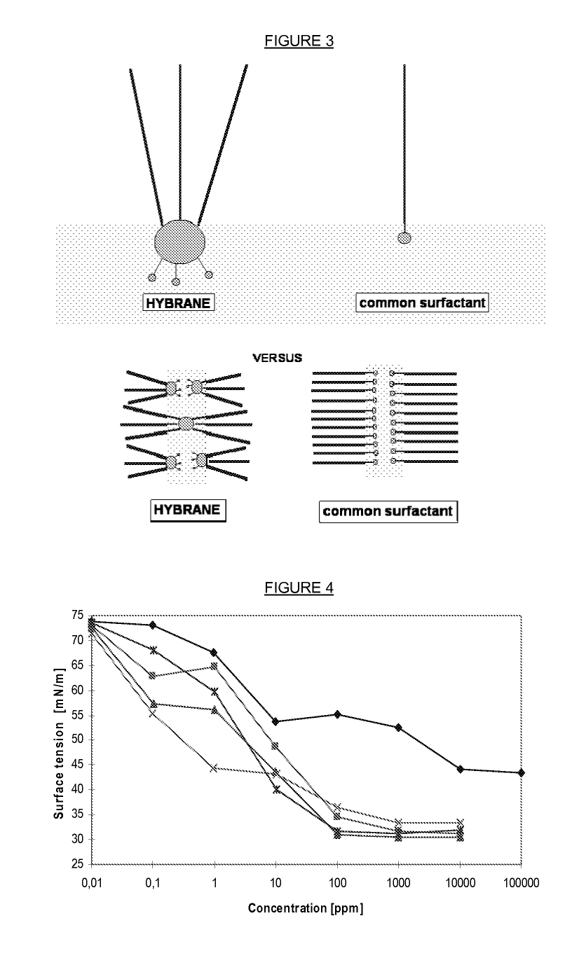

### EXAMPLES

The present invention will now be described in detail with reference to the following non limiting examples which are by way of illustration only.

### Foam Test Methods

### Test Method 1 (Simple Oil-Water Foam Test)

A 100 ml cylinder (of diameter 32 mm) is filled with 50 g of demineralised water. An amount (1000 ppm unless otherwise indicated) of the foaming agent to be tested is added to the cylinder. The mixture is shaken by hand for 30 seconds and if more than 5 ml of foam is visible for 10 seconds, the test sample is deemed to have passed and is considered suitable as a foamer.

### Test Method 2 (More Harsh Oil-Brine Foam Test)

A 1000 ml measuring cylinder is filled with 200 g sodium chloride solution (>100 g NaCl/L) of ±95° C. An amount (20 g) of condensate mixture (a mixture of alkenes like hexane) is added followed by an amount (5000 ppm unless otherwise stated) of the foaming agent to be tested. The mixture is swirled by hand for 10 seconds and the nitrogen is bubbled through the mixture at a flow rate of 50 litres per hour. The time taken for a foam to build up to a volume of 1000 ml is measured, this is denoted herein as “Foaming time” (or FT). The nitrogen bubbling is stopped and the time taken for the volume of foam to decrease to 600 ml is measured, which is denoted herein as “Foam life time” (or LT).

Various performance indicators are used herein to indicate the foam-producing capabilities of the samples tested in Test Method 2 are given below:

| Foaming time/s (denoted by ‘FT’) | Evaluation indicator |
|---|---|
| <100 s | + |
| from 100 s to 150 s | +/− |
| >150 s | − |

| Foam life-time/s (denoted by ‘LT’) | Evaluation indicator |
|---|---|
| >150 s | + |
| from 50 s to 150 s | +/− |
| <50 s | − |

### Example 1

The results of various hyperbranched polyester amides in Test Method 2 are given in the table below:

| | | | | Evaluation | Evaluation | | | Ratio polar |
|---|---|---|---|---|---|---|---|---|
| | | | | of Foaming | of Foam | Polar | Apolar | to apolar |
| Example | anhydride | end groups | Mn | Time (FT) | Lifetime (LT) | groups | groups | groups |
| Water | | | | − | − | NA | NA | NA |
| HPAx1 | H | EO2000 | 42000 | − | +/− | 6 | 12 | 0.50 |
| | | morpholine | | | | | | |
| | | laurate | | | | | | |
| HPAx2 | P | EO2000 | 6900 | − | + | 2 | 16 | 0.13 |
| | | laurate | | | | | | |
| HPAx3 | D | EO2000 | 17000 | + | + | 7 | 11 | 0.64 |
| HPAx4 | S | EO2000 | 25000 | + | + | 31 | 4 | 7.75 |
| | | stearate | | | | | | |
| HPAx5 | S | EO2000 | 15000 | − | + | 19 | 10 | 1.90 |
| | | oleate | | | | | | |
| HPAx6 | D | EO2000 | 17000 | + | + | 7 | 11 | 0.64 |
| HPAx7 | S | EO5000 | 16000 | − | +/− | 7 | 1 | 7.00 |
| | | oleate | | | | | | |
| HPAx8 | D | EO5000 | 38000 | + | + | 7 | 11 | 0.64 |
| HPAx9 | S | EO5000 | 6000 | − | + | 3 | 3 | 1.00 |
| | | oleate | | | | | | |
| HPAx10 | D | EO5000 | 16000 | + | + | 3 | 3 | 1.00 |
| HPAx11 | D | EO5000 | 67000 | + | + | 12 | 21 | 0.57 |

H = Hexahydrophthalic anhydride; P = phthalic anhydride; D = dodecenyl succinic anhydride; S = succinic anhydride

### Example 2

A surfactant with a Hyperbranched polyester amide structure HPAx10, which has a dodecenyl based core and has EO-5000 tails structure was tested as a foamer under different environmental factors like chloride content of the solution and addition of condensate.

This hyperbranched polyester amide is found to exhibit good foaming performance in a high chloride containing solution with condensate addition. And it will exhibit good foaming performance in condensate with addition of a high chloride containing solution. A relation was found with the cloud point of the Hyperbranched polyester amide in solutions but this is not the only factor which determines the foaming performance.

The results of testing HPAx10 in different environments is given in the tables below. The foaming performance was tested in solutions of low chloride concentration (1.6 g NaCl/L H2O) and high chloride concentration (200 g NaCl/L H2O) at different concentration of the hyperbranched polyester amide (HPAx10) and with different amounts of condensate.

Foam performance of HPAx10 in low chloride solution (1.6 g NaCl/L) with varying concentration and condensate amount.

| | | Foam | |
|---|---|---|---|
| Concentration | Condensate | Build-up | Foam Half- |
| (ppm) | (%) | Time/s (BT) | Life/s (HL) |
| 500 | 0 | 27 | >180 |
| 1000 | 0 | 27 | >180 |
| 2000 | 0 | 28 | >180 |
| 5000 | 0 | 30 | >180 |
| 500 | 5 | 33 | >180 |
| 1000 | 5 | 31 | >180 |
| 2000 | 5 | 34 | >180 |
| 5000 | 5 | 33 | >180 |
| 500 | 10 | 27 | 90 |
| 1000 | 10 | 31 | 120 |
| 2000 | 10 | 29 | 120 |
| 5000 | 10 | 29 | 125 |
| 5000 | 30 | 20 | 60 |

It is clear that in a solution of low chloride concentration HPAx10 has a good foam performance. All mixtures were clear when the hyperbranched polyester amide was added. Foam built-up very fast even in the presence of high amounts of condensate.

Foam performance of HPAx10 in a solution of high chloride concentration (200 g NaCl/L) with varying concentration and condensate amount.

| | | Foam | |
|---|---|---|---|
| Concentration | Condensate | Build-up | Foam Half- |
| (ppm) | (%) | Time/s (BT) | Life/s (HL) |
| 5000 | 0 | 38 | >180 |
| 5000 | 5 | 180 | >180 |
| 5000 | 10 | >180 | >180 |
| 5000 | 30 | >180 | >180 |

The foam performance of the hyperbranched polyester amide decreases drastically in high chloride solution. When the Hyperbranched polyester amide is added, the solution becomes hazy and sometimes becomes clear by cooling.

In order further to explore the effect of chloride concentration on HPAx10, experiments were done at constant hyperbranched polyester amide concentration and constant condensate amount. The results are shown in the following table.

Foam performance of HPAx10 with constant concentration and condensate amount but varying chloride solution.

| | | | Foam | |
|---|---|---|---|---|
| Concentration | Condensate | Brine | Build-up | Foam Half- |
| (ppm) | (%) | (g NaCl/L) | Time/s (BT) | Life/s (HL) |
| 5000 | 5 | 100 | 35 | >180 |
| 5000 | 5 | 150 | 43 | >180 |
| 5000 | 5 | 175 | 58 | >180 |
| 5000 | 5 | 200 | 180 | >180 |

It is surprising to see that 200 g NaCl/L is the limit for foam built up time and a stable foam is still obtained.

### Example 3

Hyper branched polyester amides in combination with common surfactants were tested. Results in the table below show the synergy between both.

| | | active | | | |
|---|---|---|---|---|---|
| | NaCl | concentration | condensate | BT | HL |
| surfactant mixture | g/l | ppm | % | sec | sec |
| HPAx10 | 200 | 5000 | 5 | 180 | 270 |
| CAPB | 200 | 5000 | 5 | 20 | 74 |
| HPAx10 + CAPB | 200 | 5000 | 5 | 19 | 120 |
| (1:2) | | | | | |
| HPAx10 + CAPB | 200 | 5000 | 5 | 20 | 215 |
| (1:10) | | | | | |
| HPAx10 | 200 | 5000 | 90 | 70 | 600+ |
| HPAx10 + CAPB | 200 | 5000 | 90 | 26 | 600+++ |
| (1:10) | | | | | |

Experiments under extreme high condensate (a mixture of high and low boiling alkanes) conditions (90% condensate and 10% high [Cl] brine solution) were performed with HPAx10 and in combination with cocoamido propyl betaine (CAPB). The results were very good because a foam builds up very fast and is very stable (lasts longer than 600 seconds).

### Example 4

Various hyperbranched polyester amides with very different structure have been tested according to Test Method 1. The table below shows that they all show foam performance.

| | | | | | Polar | Apolar | Ratio |
|---|---|---|---|---|---|---|---|
| Name | anhyhdride | end groups | Mn | Foam | groups | groups | polar/aploar |
| Water | | | | − | / | / | / |
| HPA 1 | S | OH, laurate | 1500 | + | 6 | 0.8 | 7.50 |
| HPA 2 | S | OH, laurate | 1000 | + | 5 | 1.6 | 3.13 |
| HPA 3 | D, S | OH | 1200 | + | 4.5 | 0.5 | 9.00 |
| HPA 4 | D, S | OH | 1300 | + | 4 | 1 | 4.00 |
| HPA 5 | H | morpholine, | 3500 | + | 2 | 11 | 0.18 |
| | | EO750 | | | | | |
| HPA 6 | H | amine, EO750 | 6100 | + | 7 | 11 | 0.64 |
| HPA 7 | D | EO750 | 8500 | + | 7 | 11 | 0.64 |
| HPA 8 | D | amine | 2100 | + | 3.6 | 5 | 0.72 |
| HPA 9 | H | amine | 1600 | + | 4 | 5 | 0.80 |
| HPA 10 | D | OH | 2000 | + | 8 | 5 | 1.60 |
| HPA 11 | D, S | amine, EO750 | 6600 | + | 5.5 | 12.5 | 0.44 |
| HPA 12 | S | amine, EO750 | 5100 | + | 18 | 0 | / |
| HPA 13 | S | amine, EO750 | 10000 | + | 33 | 0 | / |
| HPA 14 | S | EO750 | 6800 | + | 18 | 0 | / |
| HPA 15 | S | EO750, DEA | 7900 | + | 21 | 0 | / |
| | | based | | | | | |
| HPA 16 | S | EO750, DEA | 7700 | + | 21 | 0 | / |
| | | based | | | | | |
| HPA 17 | S | EO750, TRIS | 17000 | + | 43 | 0 | / |
| | | based | | | | | |
| HPA 18 | S | EO750, TRIS | 7200 | + | 18 | 0 | / |
| | | based | | | | | |
| HPA 19 | S | EO2000, TRIS | 29000 | + | 33 | 0 | / |
| | | based | | | | | |
| HPA 20 | S | EO2000 | 22000 | + | 27 | 0 | / |
| HPA 21 | S | EO5000 | 52000 | + | 27 | 0 | / |
| HPA 22 | D | amine, EO750 | 5200 | + | 7 | 11 | 0.64 |
| HPA 23 | D | amine, EO750 | 6300 | + | 7 | 11 | 0.64 |
| HPA 24 | D | amine, EO750 | 7500 | + | 7 | 11 | 0.64 |
| HPA 25 | D, S | amine, EO750 | 4800 | + | 9.75 | 8.25 | 1.18 |
| HPA 26 | D, S | amine, EO750 | 5900 | + | 9.75 | 8.25 | 1.18 |
| HPA 27 | D, S | amine, EO750 | 7000 | + | 9.75 | 8.25 | 1.18 |
| HPA 28 | D, S | amine, EO750 | 4200 | + | 12.5 | 5.5 | 2.27 |
| HPA 29 | D, S | amine, EO750 | 5400 | + | 12.5 | 5.5 | 2.27 |
| HPA 30 | H | morpholine, | 3800 | + | 7 | 11 | 0.64 |
| | | EO500 | | | | | |
| HPA 31 | H | morpholine, | 3500 | + | 7 | 11 | 0.64 |
| | | EO1000 | | | | | |
| HPA 32 | H | morpholine, | 6000 | + | 7 | 11 | 0.64 |
| | | EO1000 | | | | | |
| HPA 33 | H | morpholine, | 7900 | + | 7 | 11 | 0.64 |
| | | EO1000 | | | | | |
| HPA 34 | H | morpholine, | 5500 | + | 7 | 11 | 0.64 |
| | | EO2000 | | | | | |
| HPA 35 | H | morpholine, | 5000 | + | 7 | 11 | 0.64 |
| | | EO750 | | | | | |
| HPA 36 | H | morpholine, | 5400 | + | 7 | 11 | 0.64 |
| | | EO750 | | | | | |
| HPA 37 | H | morpholine, | 6100 | + | 7 | 11 | 0.64 |
| | | EO1340 | | | | | |
| HPA 38 | H | morpholine, | 47000 | + | 6 | 12 | 0.50 |
| | | EO2000 | | | | | |
| HPA 39 | H | morpholine, | 6600 | + | 7 | 11 | 0.64 |
| | | EO2000 | | | | | |
| HPA 40 | P | laurate, EO2000 | 6900 | + | 2 | 16 | 0.13 |
| HPA 41 | S | laurate, EO500 | 3400 | + | 10 | 4 | 2.50 |
| HPA 42 | D | EO2000 | 17000 | + | 7 | 11 | 0.64 |
| HPA 43 | S | stearate, | 25000 | + | 31 | 4 | 7.75 |
| | | EO2000 | | | | | |
| HPA 44 | S | oleate, | 14000 | + | 19 | 10 | 1.90 |
| | | EO2000 | | | | | |
| HPA 45 | D | EO2000 | 17000 | + | 7 | 11 | 0.64 |
| HPA 46 | S | oleate, | 16000 | + | 7 | 1 | 7.00 |
| | | EO2000 | | | | | |
| HPA 47 | D | EO5000 | 38000 | + | 7 | 11 | 0.64 |
| HPA 48 | S | oleate, | 6100 | + | 3 | 3 | 1.00 |
| | | EO5000 | | | | | |
| HPA 49 | D | EO5000 | 16000 | + | 3 | 3 | 1.00 |
| HPA 50 | D | EO5000 | 67000 | + | 12 | 21 | 0.57 |
| HPA 51 | D, S | EO5000 | 16000 | + | 5 | 1 | 5.00 |
| HPA 52 | S | oleate, | 16000 | + | 7 | 1 | 7.00 |
| | | EO5000 | | | | | |
| HPA 53 | D | amine -Me quat, | 7500 | + | 7 | 11 | 0.64 |
| | | EO750 | | | | | |
| HPA 54 | D | amine -quat, | 6300 | + | 7 | 11 | 0.64 |
| | | EO750 | | | | | |
| HPA 55 | D | amine-betaine | 17000 | + | 22 | 42 | 0.52 |
| HPA 56 | D | amine-betaine | 33000 | + | 41 | 81 | 0.51 |
| HPA 57 | D, S | amine-Me -quat | 123000 | + | 186 | 285 | 0.65 |
| HPA 58 | S | OH, stearic | 1600 | + | 5 | 1.6 | 3.13 |
| HPA 59 | S | OH, | 1200 | + | 5 | 0 | / |
| LPA 1 | | | | + | 28.75 | 37.75 | 0.76 |
| LPA 2 | | | | + | 4 | 13 | 0.31 |

1. A foam formed from a mixture of an organic phase and an aqueous phase where the foam is stabilised by a hyperbranched polyester amide.

2. A foam as claimed in claim 1 in which the hyperbranched polyester amide is obtained from at least one building block selected from the group consisting of:
C₄₋₁₆alkenyl C₂₋₁₀dioic anhydride;
C₄₋₁₆cycloalkyl dicarboxylic acid anhydride;
C₂₋₁₀alkandioic anhydride;
combinations thereof on the same moiety; and
mixtures thereof on different moieties;
at least one branching unit selected from the group consisting of:
hydroxyl substituted di-C₁₋₈hydrocarbyl secondary amine;
combinations thereof on the same moiety; and
mixtures thereof on different moieties;
where the at least one branching unit comprises at least one end group selected from the group consisting of:
optionally neutralised carboxylic acid groups;
poly(C₂₋₄alkyenyloxy)ₙ groups optionally terminated with a C₁₋₄alkoxy;
optionally quaternized C₁₋₁₂hydrocarbo substituted amino
nitrogen containing C₃₋₁₀ rings;
secondary hydroxyl groups;
combinations thereof on the same moiety; and
mixtures thereof on different moieties.

3. A foam as claimed in claim 2 in which:
the at least one building block selected from the group consisting of:
dodecenyl succinic anhydride;
cyclohexane-1,2-dicarboxylic acid anhydride;
succinic anhydride;
combinations thereof on the same moiety; and
mixtures thereof on different moieties;
the at least one branching unit selected from the group consisting of:
diisopropanol amine; diethanol amine
trishydroxymethylene amino methane;
combinations thereof on the same moiety; and
mixtures thereof on different moieties;
where the at least one branching unit comprises at least one end group selected from the group consisting of:
carboxylic acid groups optionally neutralized with ammonia;
methoxy-terminated polyethylene glycol;
3,3′-iminobis(N,N-dimethylpropyl amine) optionally quaternized with dimethyl sulfate;
morpholine;
secondary hydroxyl groups;
combinations thereof on the same moiety; and
mixtures thereof on different moieties.

4. A foam as claimed in claim 1 which further comprises at least one other surfactant and/or synergist.

5. Use of a foaming agent comprising a hyperbranched polyester amide as described in claim 1 to stabilize a foam as claimed in any preceding claim.

6. A method for forming a foam, from a mixture of an organic phase and an aqueous phase, a foam as claimed in claim 1, where the method comprising the step of:
a) adding to the mixture a hyperbranched polyester amide as described in to form the foam.

7. A method of manufacture of a hyperbranched polyester amide for the purpose of forming, from a mixture of an aqueous phase and an organic phase, a foam as claimed in claim 1.

8. A method for extracting an organic substance from a bore hole and/or well comprising the steps of:
a) adding a hyperbranched polyester amide as described in to the bore to form, from a mixture of an aqueous phase and an organic phase, a foam as claimed in claim 1;
b) transporting the foam from the bore hole and/or well;
c) destablising the foam to separate into an organic phase and an aqueous phase; and
d) optionally collecting the organic substance.

9. A method for extracting from a gas or oil well as claimed in claim 8, in which organic substance is gas or oil.

10. Use of a hyperbranched polyester amide in a method as claimed in claim 6.

11. An organic substance obtained by a method as claimed in claim 8.
